(19) United States
(12) Patent Application Publication
(10) Pub. No.: US 2012/0190698 A1
(43) Pub. Date: Jul. 26, 2012

(54) METHODS OF PREDICTING THIOPURINE RESPONSE

(75) Inventors: Marla Dubinsky, Los Angeles, CA (US); Dermot P. McGovern, Los Angeles, CA (US)

(73) Assignee: CEDARS-SINAI MEDICAL CENTER, Los Angeles, CA (US)

(21) Appl. No.: 13/358,424

(22) Filed: Jan. 25, 2012

Related U.S. Application Data
(60) Provisional application No. 61/436,129, filed on Jan. 25, 2011.

(51) Int. Cl. A61K 31/52; A61P 35/02; A61P 1/00; C12Q 1/68; A61P 29/00

(52) U.S. Cl. 514/263.3; 435/6.11

(57) ABSTRACT

The present invention relates to methods of predicting therapeutic efficacy of thiopurines in an individual by determining the presence of one or more risk variants. In one embodiment, the effective therapeutic efficacy of thiopurines is determined by the presence of risk variants at the genetic loci of HLA-DRB1, CREM, TAGAP, PLCL1, GPX4, SBNO2, MEF2A and/or LYSMD4. In another embodiment, the risk variants are located at the genetic loci of ARL4C, IL1R2, JAK2, 19q13, CARD9, SNAPC4, and/or 8q24. In another embodiment, the individual is has been diagnosed with inflammatory bowel disease.

### CROSS-REFERENCE TO RELATED APPLICATIONS

This application claims priority to U.S. Provisional Application No. 61/436,129 filed Jan. 25, 2011, which is hereby incorporated by reference in its entirety.

### FIELD OF THE INVENTION

The present invention relates generally to medical genetics, and more specifically, to inflammatory bowel disease and methods and compositions for use in predicting therapeutic outcomes with thiopurines.

### BACKGROUND

All publications herein are incorporated by reference to the same extent as if each individual publication or patent application was specifically and individually indicated to be incorporated by reference. The following description includes information that may be useful in understanding the present invention. It is not an admission that any of the information provided herein is prior art or relevant to the presently claimed invention, or that any publication specifically or implicitly referenced is prior art.

Crohn's disease (CD) and ulcerative colitis (UC), the two common forms of idiopathic inflammatory bowel disease (IBD), are chronic, relapsing inflammatory disorders of the gastrointestinal tract. Each has a peak age of onset in the second to fourth decades of life and prevalences in European ancestry populations that average approximately 100-150 per 100,000 (D. K. Podolsky, N Engl J Med 347, 417 (2002); E. V. Loftus, Jr., Gastroenterology 126, 1504 (2004)). Although the precise etiology of IBD remains to be elucidated, a widely accepted hypothesis is that ubiquitous, commensal intestinal bacteria trigger an inappropriate, overactive, and ongoing mucosal immune response that mediates intestinal tissue damage in genetically susceptible individuals (D. K. Podolsky, N Engl J Med 347, 417 (2002)). Genetic factors play an important role in IBD pathogenesis, as evidenced by the increased rates of IBD in Ashkenazi Jews, familial aggregation of IBD, and increased concordance for IBD in monozygotic compared to dizygotic twin pairs (S. Vermeire, P. Rutgeerts, Genes Immun 6, 637 (2005)). Moreover, genetic analyses have linked IBD to specific genetic variants, especially CARD15 variants on chromosome 16q12 and the IBD5 haplotype (spanning the organic cation transporters, SLC22A4 and SLC22A5, and other genes) on chromosome 5q31 (S. Vermeire, P. Rutgeerts, Genes Immun 6, 637 (2005); J. P. Hugot et al., Nature 411, 599 (2001); Y. Ogura et al., Nature 411, 603 (2001); J. D. Rioux et al., Nat Genet 29, 223 (2001); V. D. Peltekova et al., Nat Genet 36, 471 (2004)). CD and UC are thought to be related disorders that share some genetic susceptibility loci but differ at others.

Thiopurines have been found to be useful in the treatment of inflammatory bowel disease (IBD), and may be metabolized by methylation by thiopurine methyltransferase (or TPMT). However, variation in thiopurine methyltransferase (TPMT) activity does not fully account for differences in interindividual clinical response to thiopurines in inflammatory bowel disease (IBD). Other genetic and immune biomarkers may also predict therapeutic outcomes with thiopurines.

### SUMMARY OF THE INVENTION

Various methods herein include a method of predicting responsiveness to thiopurine treatment in an individual, comprising obtaining a sample from the individual, assaying the sample to determine the presence or absence of one or more risk variants at the HLA-DRB1, CREM, TAGAP, PLCL1, GPX4, SBNO2, MEF2A and/or LYSMD4 genetic loci, predicting responsiveness to thiopurine treatment based on the presence of one or more risk variants at the HLA-DRB1, CREM, TAGAP, PLCL1, GPX4, SBNO2, MEF2A and/or LYSMD4 genetic loci. In another embodiment, the individual has been diagnosed with inflammatory bowel disease. In another embodiment, the individual is a child. In another embodiment, the risk variants include SEQ. ID. NO.: 1, SEQ. ID. NO.: 2, SEQ. ID. NO.: 3, SEQ. ID. NO.: 4, and/or SEQ. ID. NO.: 5. In another embodiment, the presence of five or more said risk variants presents a greater probability of responsiveness to thiopurine treatment than the presence of four, three, two, one or none of said risk variants; the presence of four said risk variants presents a greater probability of responsiveness to thiopurine treatment than the presence of three, two, one or none of said risk variants; the presence of three risk variants presents a greater probability of responsiveness to thiopurine treatment than the presence of two, one or none of said risk variants; the presence of two risk variants presents a greater probability of responsiveness to thiopurine treatment than the presence of one or none of said risk variants; and the presence of one risk variant presents a greater probability of responsiveness to thiopurine treatment than the presence of none of said risk variants. In another embodiment, the individual has been diagnosed with ulcerative colitis. In another embodiment, the sample further comprises a high expression relative to a normal subject of pANCA. In another embodiment, the individual has been diagnosed with acute lymphoblastic leukemia and/or an autoimmune disorder. In another embodiment, the individual is an organ transplant recipient.

Other embodiments include a method of treating a disease in an individual, comprising determining the presence of one or more risk variants in the individual at the HLA-DRB1, CREM, TAGAP, PLCL1, GPX4, SBNO2, MEF2A and/or LYSMD4 genetic loci, and administering a therapeutically effective dosage to the individual of a composition comprising thiopurine, or a pharmaceutical equivalent, analog, derivative, and/or salt thereof. In another embodiment, the risk variants include SEQ. ID. NO.: 1, SEQ. ID. NO.: 2, SEQ. ID. NO.: 3, SEQ. ID. NO.: 4, SEQ. ID. NO.: 5, SEQ. ID. NO.: 6, SEQ. ID. NO.: 7, SEQ. ID. NO.: 8, SEQ. ID. NO.: 9, SEQ. ID. NO.: 10 and/or SEQ. ID. NO.: 11. In another embodiment, the disease is inflammatory bowel disease. In another embodiment, the disease is acute lymphoblastic leukemia and/or an autoimmune disorder. In another embodiment, the sample further comprises a high expression relative to a normal subject of pANCA.

Other embodiments include a method of predicting responsiveness to thiopurine treatment in an individual, comprising obtaining a sample from the individual, assaying the sample to determine the presence or absence of one or more risk variants at the ARL4C, IL1R2, JAK2, 19q13, TAGAP, CARD9, SNAPC4, 8q24 and/or HLA-DRB1 genetic loci, predicting responsiveness to thiopurine treatment based on the presence of one or more risk variants at the ARL4C, IL1R2, JAK2, 19q13, TAGAP, CARD9, SNAPC4, 8q24 and/or HLA-DRB1 genetic loci. In another embodiment, the individual has been diagnosed with inflammatory bowel disease. In another embodiment, the individual is a child. In another embodiment, the risk variants include SEQ. ID. NO.: 1, SEQ. ID. NO.: 3, SEQ. ID. NO.: 6, SEQ. ID. NO.: 7, SEQ. ID. NO.: 8, SEQ. ID. NO.: 9, SEQ. ID. NO.: 10 and/or SEQ. ID. NO.: 11. In another embodiment, the individual is male.

Other features and advantages of the invention will become apparent from the following detailed description, taken in conjunction with the accompanying drawings, which illustrate, by way of example, various embodiments of the invention.

### DESCRIPTION OF THE INVENTION

All references cited herein are incorporated by reference in their entirety as though fully set forth. Unless defined otherwise, technical and scientific terms used herein have the same meaning as commonly understood by one of ordinary skill in the art to which this invention belongs. Singleton et al., Dictionary of Microbiology and Molecular Biology 3rd ed., J. Wiley & Sons (New York, N.Y. 2001); March, Advanced Organic Chemistry Reactions, Mechanisms and Structure 5th ed., J. Wiley & Sons (New York, NY 2001); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described.

“IBD” as used herein is an abbreviation of inflammatory bowel disease.

“CD” as used herein is an abbreviation of Crohn's Disease.

“GWAS” as used herein is an abbreviation for genome wide association study.

“TPMT” as used herein is an abbreviation for thiopurine methyltransferase.

“HLA-DRB1” as used herein is an abbreviation for HLA class II histocompatibility antigen, DRB 1-9 beta chain.

“CREM” as used herein is an abbreviation for cAMP-responsive element modulator.

“TAGAP” as used herein is an abbreviation for T-cell activation RhoGTPase activating protein.

“PLCL1” as used herein is an abbreviation for phospholipase C-like 1.

“GPX4” as used herein is an abbreviation for glutathione peroxidase 4.

“SBNO2” as used herein is an abbreviation for strawberry notch homolog 2.

“MEF2A” as used herein is an abbreviation for myocyte enhancer factor 2A.

“LYSMD4” as used herein is an abbreviation for LysM, putative peptidoglycan-binding, domain containing 4.

Various single nucleotide polymorphisms (SNPs) are used herein. Examples of SNPs rs2516049, rs3936503, rs212388, rs10196612, rs2024092, rs4663142, rs2310173, rs10758669, rs736289, rs4077515, and rs6651252, are provided herein as SEQ. ID. NO.: 1, SEQ. ID. NO.: 2, SEQ. ID. NO.: 3, SEQ. ID. NO.: 4, SEQ. ID. NO.: 5, SEQ. ID. NO.: 6, SEQ. ID. NO.: 7, SEQ. ID. NO.: 8, SEQ. ID. NO.: 9, SEQ. ID. NO.: 10, and SEQ. ID. NO.: 11, respectively.

As used herein, the term “risk variant” means a variant, or marker, that is indicative of a likelihood of successful thiopurine-induced corticosteroid-free remission in the individual being treated with thiopurine.

“Thiopurine drugs” as used herein, are purine antimetabolites widely used in the treatment of acute lymphoblastic leukemia, autoimmune disorders, and organ transplant recipients. Similarly, “thiopurine treatment” as used herein means treating a condition or disease in an individual through the administration of thiopurine, or a pharmaceutical equivalent, analog, derivative, and/or salt thereof, to the individual being treated.

As used herein, the term “biological sample” means any biological material from which nucleic acid molecules can be prepared. As non-limiting examples, the term material encompasses whole blood, plasma, saliva, cheek swab, or other bodily fluid or tissue that contains nucleic acid.

As disclosed herein, the inventors tested associations of known IBD susceptibility loci and pharmacogenetic genome-wide association study (GWAS)-identified loci, as well as clinical and immune phenotypes, with thiopurine-induced corticosteroid-free remission in IBD, and developed a predictive model of remission. Corticosteroid-free remission at 26 weeks after thiopurine initiation was defined using the Harvey Bradshaw Index (HBI) for Crohn's disease (CD) and partial Mayo score for ulcerative colitis (UC). Serum was assayed for ASCA IgA and IgG, anti-OmpC, anti-CBirl, 12, and pANCA using ELISA. Clinical phenotypes included age, gender, IBD subtype (CD versus UC), disease duration at thiopurine initiation, and age at diagnosis. Genotyping was performed using Illumina technology. Univariate analyses tested associations of phenotype and genotype with remission. Stepwise logistic regression was performed to build predictive models.

As further disclosed herein, corticosteroid-free remission occurred in 56 of 122 subjects (45.9%) at week 26. Female gender (OR=0.37; 95% CI: 0.18-0.77; P=0.011) and pANCA (OR=0.23; 95% CI: 0.06-0.87; P=0.049) were negatively associated with corticosteroid-free remission at 26 weeks. Five known IBD susceptibility loci were associated with corticosteroid-free remission (P<0.05) (Table 1 herein). A single nucleotide polymorphism (SNP) at 15q31 tagging MEF2A (macrophage differentiation) and LYSMD4 (peptidoglycan binding) met the criteria for nominal association at the genome wide level for remission (OR=9.5; P=3E-05). The most predictive model of remission included the previously identified HLA-DRB1 locus (rs2516049), 7 novel “pharmacogenetic” GWAS loci, pANCA, disease duration, and a diagnosis of UC with an R-squared of 0.884, area under the curve [AUC] of 0.985, sensitivity of 0.929, specificity of 0.919, accuracy of 0.826, and positive likelihood ratio of 11.45. The probability of remission increased 7.3-fold when the number of predictors increased from 0-4 to 5-7 (95% CI: 2.43-21.66; P=0.0004). The combination of genotype with clinical and immune phenotypes is most predictive of corticosteroid-free remission after thiopurine initiation. Defining predictors of therapeutic efficacy to thiopurines allows identification of patients who will benefit most from this class of therapy, contributing to a more individualized approach to therapy.

In one embodiment, the present invention provides a method of predicting a therapeutic efficacy of thiopurines in an individual, by determining the presence or absence of one or more risk variants at the HLA-DRB 1, CREM, TAGAP, PLCL 1, GPX4 and/or SBNO2 genetic loci, wherein the presence of one or more risk variants at the HLA-DRB1, CREM, TAGAP, PLCL1, GPX4 and/or SBNO2 genetic loci is indicative of an effective therapeutic efficacy of thiopurines in the individual. In another embodiment, the risk variants are located at the genetic loci of ARL4C, IL1R2, JAK2, 19q13, CARD9, SNAPC4, and/or 8q24. In another embodiment, the individual has inflammatory bowel disease (IBD). In another embodiment, the effective therapeutic efficacy of thiopurines is a thiopurine-induced corticosteroid-free remission in IBD. In another embodiment, the one or more risk variants are associated with one or more antibody markers. In another embodiment, the one or more risk variants are listed in Table 1 herein. In another embodiment, the individual is a child.

In another embodiment, the present invention provides a method of treating IBD in an individual, comprising determining the presence of one or more genetic risk variants at the genetic loci of ARL4C, IL1R2, JAK2, 19q13, CARD9, SNAPC4, 8q24, HLA-DRB1, CREM, TAGAP, PLCL1, GPX4 and/or SBNO2, and then treating the individual by administering a composition comprising thiopurine, or a pharmaceutical equivalent, analog, derivative, and/or salt thereof.

A variety of methods can be used to determine the presence or absence of a variant allele or haplotype. As an example, enzymatic amplification of nucleic acid from an individual may be used to obtain nucleic acid for subsequent analysis. The presence or absence of a variant allele or haplotype may also be determined directly from the individual's nucleic acid without enzymatic amplification.

Analysis of the nucleic acid from an individual, whether amplified or not, may be performed using any of various techniques. Useful techniques include, without limitation, polymerase chain reaction based analysis, sequence analysis and electrophoretic analysis. As used herein, the term “nucleic acid” means a polynucleotide such as a single or double-stranded DNA or RNA molecule including, for example, genomic DNA, cDNA and mRNA. The term nucleic acid encompasses nucleic acid molecules of both natural and synthetic origin as well as molecules of linear, circular or branched configuration representing either the sense or antisense strand, or both, of a native nucleic acid molecule.

The presence or absence of a variant allele or haplotype may involve amplification of an individual's nucleic acid by the polymerase chain reaction. Use of the polymerase chain reaction for the amplification of nucleic acids is well known in the art (see, for example, Mullis et al. (Eds.), The Polymerase Chain Reaction, Birkhauser, Boston, (1994)).

A TaqmanB allelic discrimination assay available from Applied Biosystems may be useful for determining the presence or absence of a variant allele. In a TaqmanB allelic discrimination assay, a specific, fluorescent, dye-labeled probe for each allele is constructed. The probes contain different fluorescent reporter dyes such as FAM and VICTM to differentiate the amplification of each allele. In addition, each probe has a quencher dye at one end which quenches fluorescence by fluorescence resonant energy transfer (FRET). During PCR, each probe anneals specifically to complementary sequences in the nucleic acid from the individual. The 5′ nuclease activity of Taq polymerase is used to cleave only probe that hybridize to the allele. Cleavage separates the reporter dye from the quencher dye, resulting in increased fluorescence by the reporter dye. Thus, the fluorescence signal generated by PCR amplification indicates which alleles are present in the sample. Mismatches between a probe and allele reduce the efficiency of both probe hybridization and cleavage by Taq polymerase, resulting in little to no fluorescent signal. Improved specificity in allelic discrimination assays can be achieved by conjugating a DNA minor grove binder (MGB) group to a DNA probe as described, for example, in Kutyavin et al., “3′-minor groove binder-DNA probes increase sequence specificity at PCR extension temperature, “Nucleic Acids Research 28:655-661 (2000)). Minor grove binders include, but are not limited to, compounds such as dihydrocyclopyrroloindole tripeptide (DPI,).

Sequence analysis also may also be useful for determining the presence or absence of a variant allele or haplotype.

Restriction fragment length polymorphism (RFLP) analysis may also be useful for determining the presence or absence of a particular allele (Jarcho et al. in Dracopoli et al., Current Protocols in Human Genetics pages 2.7.1-2.7.5, John Wiley & Sons, New York; Innis et al.,(Ed.), PCR Protocols, San Diego: Academic Press, Inc. (1990)). As used herein, restriction fragment length polymorphism analysis is any method for distinguishing genetic polymorphisms using a restriction enzyme, which is an endonuclease that catalyzes the degradation of nucleic acid and recognizes a specific base sequence; generally a palindrome or inverted repeat. One skilled in the art understands that the use of RFLP analysis depends upon an enzyme that can differentiate two alleles at a polymorphic site.

Allele-specific oligonucleotide hybridization may also be used to detect a disease-predisposing allele. Allele-specific oligonucleotide hybridization is based on the use of a labeled oligonucleotide probe having a sequence perfectly complementary, for example, to the sequence encompassing a disease-predisposing allele. Under appropriate conditions, the allele-specific probe hybridizes to a nucleic acid containing the disease-predisposing allele but does not hybridize to the one or more other alleles, which have one or more nucleotide mismatches as compared to the probe. If desired, a second allele-specific oligonucleotide probe that matches an alternate allele also can be used. Similarly, the technique of allele-specific oligonucleotide amplification can be used to selectively amplify, for example, a disease-predisposing allele by using an allele-specific oligonucleotide primer that is perfectly complementary to the nucleotide sequence of the disease-predisposing allele but which has one or more mismatches as compared to other alleles (Mullis et al., supra, (1994)). One skilled in the art understands that the one or more nucleotide mismatches that distinguish between the disease-predisposing allele and one or more other alleles are preferably located in the center of an allele-specific oligonucleotide primer to be used in allele-specific oligonucleotide hybridization. In contrast, an allele-specific oligonucleotide primer to be used in PCR amplification preferably contains the one or more nucleotide mismatches that distinguish between the disease-associated and other alleles at the 3′ end of the primer.

A heteroduplex mobility assay (HMA) is another well known assay that may be used to detect a SNP or a haplotype. HMA is useful for detecting the presence of a polymorphic sequence since a DNA duplex carrying a mismatch has reduced mobility in a polyacrylamide gel compared to the mobility of a perfectly base-paired duplex (Delwart et al., Science 262:1257-1261 (1993); White et al., Genomics 12:301-306 (1992)).

The technique of single strand conformational, polymorphism (SSCP) also may be used to detect the presence or absence of a SNP and/or a haplotype (see Hayashi, K., Methods Applic. 1:34-38 (1991)). This technique can be used to detect mutations based on differences in the secondary structure of single-strand DNA that produce an altered electrophoretic mobility upon non-denaturing gel electrophoresis. Polymorphic fragments are detected by comparison of the electrophoretic pattern of the test fragment to corresponding standard fragments containing known alleles.

Denaturing gradient gel electrophoresis (DGGE) also may be used to detect a SNP and/or a haplotype. In DGGE, double-stranded DNA is electrophoresed in a gel containing an increasing concentration of denaturant; double-stranded fragments made up of mismatched alleles have segments that melt more rapidly, causing such fragments to migrate differently as compared to perfectly complementary sequences (Sheffield et al., “Identifying DNA Polymorphisms by Denaturing Gradient Gel Electrophoresis” in Innis et al., supra, 1990).

Other molecular methods useful for determining the presence or absence of a SNP and/or a haplotype are known in the art and useful in the methods of the invention. Other well-known approaches for determining the presence or absence of a SNP and/or a haplotype include automated sequencing and RNAase mismatch techniques (Winter et al., Proc. Natl. Acad. Sci. 82:7575-7579 (1985)). Furthermore, one skilled in the art understands that, where the presence or absence of multiple alleles or haplotype(s) is to be determined, individual alleles can be detected by any combination of molecular methods. See, in general, Birren et al. (Eds.) Genome Analysis: A Laboratory Manual Volume 1 (Analyzing DNA) New York, Cold Spring Harbor Laboratory Press (1997). In addition, one skilled in the art understands that multiple alleles can be detected in individual reactions or in a single reaction (a “multiplex” assay). In view of the above, one skilled in the art realizes that the methods of the present invention for diagnosing or predicting susceptibility to or protection against CD in an individual may be practiced using one or any combination of the well known assays described above or another art-recognized genetic assay.

The present invention is also directed to a kit to predict therapeutic remission to thiopurines. The kit is an assemblage of materials or components, including at least one of the inventive compositions. The exact nature of the components configured in the inventive kit depends on its intended purpose. For example, some embodiments are configured for the purpose of treating inflammatory bowel disease. In one embodiment, the kit is configured particularly for the purpose of treating mammalian subjects. In another embodiment, the kit is configured particularly for the purpose of treating human subjects. In further embodiments, the kit is configured for veterinary applications, treating subjects such as, but not limited to, farm animals, domestic animals, and laboratory animals.

Instructions for use may be included in the kit. “Instructions for use” typically include a tangible expression describing the technique to be employed in using the components of the kit to effect a desired outcome, such as to predict therapeutic remission to thiopurines, or to treat IBD, or to perform an organ transplantation. Optionally, the kit also contains other useful components, such as, diluents, buffers, pharmaceutically acceptable carriers, syringes, catheters, applicators, pipetting or measuring tools, bandaging materials or other useful paraphernalia as will be readily recognized by those of skill in the art.

The materials or components assembled in the kit can be provided to the practitioner stored in any convenient and suitable ways that preserve their operability and utility. For example the components can be in dissolved, dehydrated, or lyophilized form; they can be provided at room, refrigerated or frozen temperatures. The components are typically contained in suitable packaging material(s). As employed herein, the phrase “packaging material” refers to one or more physical structures used to house the contents of the kit, such as inventive compositions and the like. The packaging material is constructed by well known methods, preferably to provide a sterile, contaminant-free environment. As used herein, the term “package” refers to a suitable solid matrix or material such as glass, plastic, paper, foil, and the like, capable of holding the individual kit components. The packaging material generally has an external label which indicates the contents and/or purpose of the kit and/or its components.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

### EXAMPLES

The following examples are provided to better illustrate the claimed invention and are not to be interpreted as limiting the scope of the invention. To the extent that specific materials are mentioned, it is merely for purposes of illustration and is not intended to limit the invention. One skilled in the art may develop equivalent means or reactants without the exercise of inventive capacity and without departing from the scope of the invention.

### Example 1

### Therapeutic Remission to Thiopurines in IBD

As disclosed herein, the inventors tested associations of known IBD susceptibility loci and novel “pharmacogenetic” genome-wide association study (GWAS)-identified loci, as well as clinical and immune phenotypes, with thiopurine-induced corticosteroid-free remission in IBD, and developed a predictive model of remission. Corticosteroid-free remission at 26 weeks after thiopurine initiation was defined using the Harvey Bradshaw Index (HBI) for Crohn's disease (CD) and partial Mayo score for ulcerative colitis (UC). Serum was assayed for ASCA IgA and IgG, anti-OmpC, anti-CBirl, 12, and pANCA using ELISA. Clinical phenotypes included age, gender, IBD subtype (CD versus UC), disease duration at thiopurine initiation, and age at diagnosis. Genotyping was performed using Illumina technology. Univariate analyses tested associations of phenotype and genotype with remission. Stepwise logistic regression was performed to build predictive models.

As further disclosed herein, corticosteroid-free remission occurred in 56 of 122 subjects (45.9%) at week 26. Female gender (OR=0.37; 95% CI: 0.18-0.77; P=0.011) and pANCA (OR=0.23; 95% CI: 0.06-0.87; P=0.049) were negatively associated with corticosteroid-free remission at 26 weeks. Five known IBD susceptibility loci were associated with corticosteroid-free remission (P<0.05) (Table 1 herein). A single nucleotide polymorphism (SNP) at 15₈31) tagging MEF2A (macrophage differentiation) and LYSMD4 (peptidoglycan binding) met the criteria for nominal association at the genome wide level for remission (OR=9.5; P=3E-05). The most predictive model of remission included the previously identified HLA-DRB1 locus (rs2516049; SEQ. ID. NO.: 1), 7 pharmacogenetic GWAS loci, pANCA, disease duration, and a diagnosis of UC with an R-squared of 0.884, area under the curve [AUC] of 0.985, sensitivity of 0.929, specificity of 0.919, accuracy of 0.826, and positive likelihood ratio of 11.45. The probability of remission increased 7.3-fold when the number of predictors increased from 0-4 to 5-7 (95% CI: 2.43-21.66; P=0.0004). The combination of genotype with clinical and immune phenotypes is most predictive of corticosteroid-free remission after thiopurine initiation. Defining predictors of therapeutic efficacy to thiopurines allows identification of patients who will benefit most from this class of therapy, contributing to a more individualized approach to therapy.

### Example 2

TABLE 1
SNPs associated with corticosteroid-free remission
with thiopurines at week 26

| SNP | Chromosome | P Value | OR | Gene of Interest |
|---|---|---|---|---|
| (SEQ. ID. NO.: 1) | 6 | 0.0033 | 3.35 | HLA-DRB1 |
| rs2516049 | | | | |
| (SEQ. ID. NO.: 2) | 10 | 0.019 | 10.40 | CREM |
| rs3936503 | | | | |
| (SEQ. ID. NO.: 3) | 6 | 0.042 | 2.04 | TAGAP |
| rs212388 | | | | |
| (SEQ. ID. NO.: 4) | 2 | 0.043 | 0.50 | PLCL1 |
| rs10196612 | | | | |
| (SEQ. ID. NO.: 5) | 19 | 0.044 | 0.44 | GPX4, SBNO2 |
| rs2024092 | | | | |

### Example 3

### Genotyping

Genotyping performed at the Medical Genetics Institute at Cedars-Sinai Medical Center
Using Illumina Human610 and OmniExpress chips for CD samples
Using Illumina HumanCNV370 and OmniExpress chips for UC samples

191,264 SNPs were common among platforms, passed quality control, and were included in the analyses

Principal components analysis using Eigenstrat was conducted to examine population stratification

### Example 4

### Univariate Associations

1) Genetic loci
Known IBD susceptibility SNPs (GWA significance) from:
   1) adult CD meta GWAS (71 loci)
   2) adult UC meta GWAS (47 loci)
   3) pediatric IBD GWAS (2 loci)
Pharmacogenetic based GWAS SNPs
   Single SNP associations

2) Demographic and clinical phenotype data
Student's t-test for continuous variables
Fisher exact test for categorical variables

### Example 5

### Predictive Models

1. Multiple Logistic Regression models of remission:
Model I: Clinical Only—Including Age, Gender, IBD subtype: UC vs. CD, Disease duration, TPMT activity, Starting dose AZA≧2.5 mg/kg/day or equivalent.
Model II: Genetics only—Including known IBD Susceptibility SNPs
(“Top Hits” p<0.05) & SNPs from Pharmacogenetic GWAS (p<1×10-5)
Model III: Genetics +Clinical (Final model)

2. Clinical Utility Measures: sensitivity, specificity, AUC, positive likelihood ratio

### Example 6

TABLE 2
Clinical phenotypic associations with therapeutic outcomes to
thiopurines (week 26)

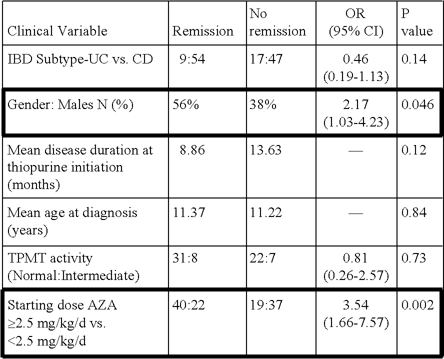

### Example 7

TABLE 3
Known IBD susceptibility loci associated with
remission at week 26 (p < 0.05)

| Gene(s) of | | CD or | | | |
|---|---|---|---|---|---|
| Interest | Chromosome | UC | SNP | OR | P value |
| TAGAP | 6 | CD | rs212388 | 2.48 | 0.003 |
| 19q13 | 19 | CD, UC | rs736289 | 0.45 | 0.008 |
| CARD9, | 9 | CD | rs4077515 | 0.53 | 0.026 |
| SNAPC4 | | | | | |
| HLA-DRB1 | 6 | CD, UC | rs2516049 | 1.90 | 0.036 |
| JAK2 | 9 | CD, UC | rs10758669 | 1.78 | 0.043 |
| IL1R2 | 2 | UC | rs2310173 | 2.00 | 0.046 |
| 8q24 | 8 | CD | rs6651252 | 0.41 | 0.047 |

### Example 8

TABLE 4
Model 1 results, Clinical phenotype only

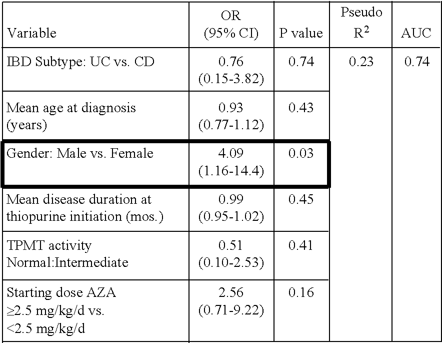

### Example 9

TABLE 5
Model II results, Genotype only

GWAS p < 1 × 10\hat{ }-5
Top hits p < 0.05

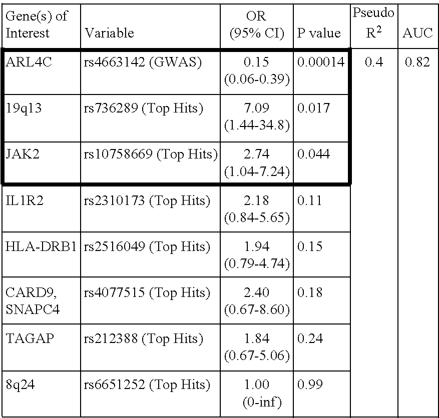

### Example 10

TABLE 6
Model III, Genotype + Clinical Phenotype

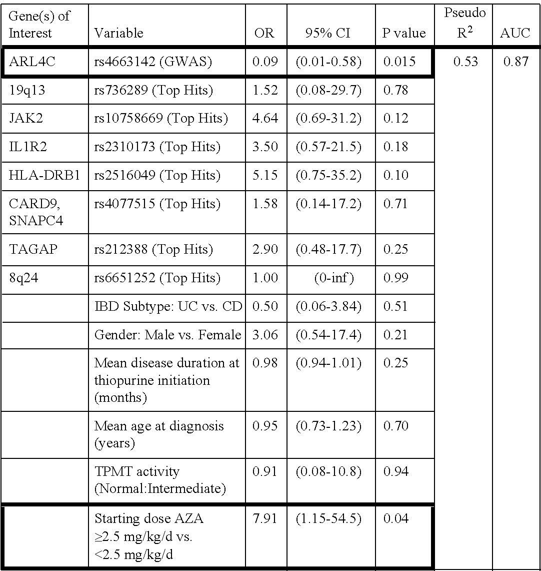

### Example 11

TABLE 7
Model III (genotype + clinical): Demonstration of diagnostic utility for
remission to thiopurines

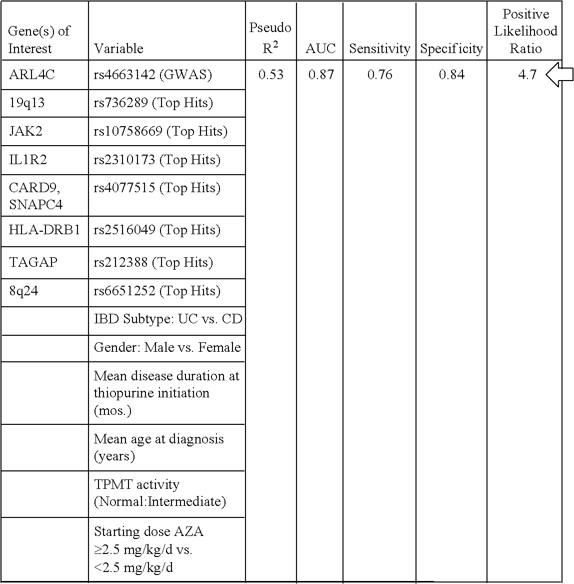

### Example 12

### Overall

7 known IBD susceptibility loci (“top hits”) were significantly associated with thiopurine-induced corticosteroid-free remission at week 26, with p<0.05. Additionally, the ARL4C locus met nominal genome-wide significance for association with remission at week 26 (p=3.11 E-06). The model combining clinical phenotype and genotype was the most predictive of corticosteroid-free remission (LR:4.7), and TPMT activity was not associated with corticosteroid-free remission.

### Example 13

TABLE 8
Description of genetic loci

| Gene(s) of | | | |
|---|---|---|---|
| Interest | SNP | Details | Role |
| ARL4C | rs4663142 | Member of ADP-ribosylation | Lymphoid/ |
| 2q37/IBD | (GWAS) | factor family of GTP-binding | myeloid |
| | | proteins. May be involved in | differentiation |
| | | lymphoid/myeloid | & intracellular |
| | | differentiation and | transport |
| | | intracellular transport. | |
| | | Expressed in T cells, NK | |
| | | cells, and dendritic cells. | |
| IL1R2 | rs2310173 | Cytokine receptor belonging | Pro- |
| 2q12/UC | (Top Hit) | to interleukin 1 receptor | inflammatory |
| | | family. Acts as a decoy | |
| | | receptor to inhibit the activity | |
| | | of its ligands (IL1a, IL1b, | |
| | | IL1R1) | |
| JAK2 | rs10758669 | Signal Transducers and | Signal |
| 9p24/IBD | (Top Hit) | Activators of Transcription | transduction |
| | | (STAT)-Janus kinase (JAK) | (e.g., IFN-g) |
| | | pathway controls signal | |
| | | transduction between cell | |
| | | surface receptors & the | |
| | | nucleus | |
| 19q13 | rs736289 | Associated with CD, UC | Unknown |
| IBD | (Top Hit) | | |
| TAGAP | rs212388 | T cell activation Rho GTPase | T cell |
| 6q25/CD | (Top Hit) | activating protein. Shared | activation |
| | | risk locus in celiac disease | |
| CARD9, | rs4077515 | Caspase-associated | Cell |
| SNAPC4 | (Top Hit) | recruitment domain family, | apoptosis, |
| 9q34/CD | | member 9. Small nuclear | RNA Pol |
| | | RNA activating complex, | |
| | | polypeptide 4, required for | |
| | | RNA polymerase II/III | |
| | | snRNA | |
| 8q24 | rs6651252 | Associated with CD | Unknown |
| CD | (Top Hit) | | |
| HLA- | rs2516049 | HLA class II | Antigen |
| DRB1 | (Top Hit) | histocompatibility antigen, | presentation |
| 6p21/IBD | | encodes the most prevalent | |
| | | beta subunit of HLA-DR. | |
| | | Presents peptides derived | |
| | | from extracellular proteins | |

While the description above refers to particular embodiments of the present invention, it should be readily apparent to people of ordinary skill in the art that a number of modifications may be made without departing from the spirit thereof. The presently disclosed embodiments are, therefore, to be considered in all respects as illustrative and not restrictive.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.

Many variations and alternative elements have been disclosed in embodiments of the present invention. Still further variations and alternate elements will be apparent to one of skill in the art. Among these variations, without limitation, are the selection of constituent modules for the inventive compositions, and the diseases and other clinical conditions that may be diagnosed, prognosed or treated therewith. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

1. A method of predicting responsiveness to thiopurine treatment in an individual, comprising:
obtaining a sample from the individual;
assaying the sample to determine the presence or absence of one or more risk variants at the HLA-DRB1, CREM, TAGAP, PLCL1, GPX4, SBNO2, MEF2A and/or LYSMD4 genetic loci; and
predicting responsiveness to thiopurine treatment based on the presence of one or more risk variants at the HLA-DRB1, CREM, TAGAP, PLCL1, GPX4, SBNO2, MEF2A and/or LYSMD4 genetic loci.

2. The method of claim 1, wherein the individual has been diagnosed with inflammatory bowel disease.

3. The method of claim 1, wherein the individual is a child.

4. The method of claim 1, wherein the risk variants comprise SEQ. ID. NO.: 1, SEQ. ID. NO.: 2, SEQ. ID. NO.: 3, SEQ. ID. NO.: 4, and/or SEQ. ID. NO.: 5.

5. The method of claim 1, wherein the presence of five or more of said risk variants presents a greater probability of responsiveness to thiopurine treatment than the presence of four, three, two, one or none of said risk variants; the presence of four said risk variants presents a greater probability of responsiveness to thiopurine treatment than the presence of three, two, one or none of said risk variants; the presence of three risk variants presents a greater probability of responsiveness to thiopurine treatment than the presence of two, one or none of said risk variants; the presence of two risk variants presents a greater probability of responsiveness to thiopurine treatment than the presence of one or none of said risk variants; and the presence of one risk variant presents a greater probability of responsiveness to thiopurine treatment than the presence of none of said risk variants.

6. The method of claim 1, wherein the individual has been diagnosed with ulcerative colitis.

7. The method of claim 1, wherein the sample further comprises a high expression relative to a normal subject of pANCA.

8. The method of claim 1, wherein the individual has been diagnosed with acute lymphoblastic leukemia and/or an autoimmune disorder.

9. The method of claim 1, wherein the individual is an organ transplant recipient.

10. A method of treating a disease in an individual, comprising:
determining the presence of one or more risk variants in the individual at the genetic loci of ARL4C, IL1R2, JAK2, 19q13, CARD9, SNAPC4, 8q24, HLA-DRB1, CREM, TAGAP, PLCL1, GPX4, SBNO2, MEF2A and/or LYSMD4; and
administering a therapeutically effective dosage to the individual of a composition comprising thiopurine, or a pharmaceutical equivalent, analog, derivative, and/or salt thereof

11. The method of claim 10, wherein the risk variants comprise SEQ. ID. NO.: 1, SEQ. ID. NO.: 2, SEQ. ID. NO.: 3, SEQ. ID. NO.: 4, SEQ. ID. NO.: 5, SEQ. ID. NO.: 6, SEQ. ID. NO.: 7, SEQ. ID. NO.: 8, SEQ. ID. NO.: 9, SEQ. ID. NO.: 10 and/or SEQ. ID. NO.: 11.

12. The method of claim 10, wherein the disease is inflammatory bowel disease.

13. The method of claim 10, wherein the disease is acute lymphoblastic leukemia and/or an autoimmune disorder.

14. The method of claim 10, wherein the individual demonstrates a high expression relative to a normal subject of pANCA.

15. The method of claim 10, wherein the individual is male.

16. A method of predicting responsiveness to thiopurine treatment in an individual, comprising:
obtaining a sample from the individual;
assaying the sample to determine the presence or absence of one or more risk variants at the ARL4C, IL1R2, JAK2, 19q13, TAGAP, CARD9, SNAPC4, 8q24 and/or HLA-DRB1 genetic loci; and
predicting responsiveness to thiopurine treatment based on the presence of one or more risk variants at the ARL4C, IL1R2, JAK2, 19q13, TAGAP, CARD9, SNAPC4, 8q24 and/or HLA-DRB1 genetic loci.

17. The method of claim 16, wherein the individual has been diagnosed with inflammatory bowel disease.

18. The method of claim 16, wherein the individual is a child.

19. The method of claim 16, wherein the risk variants comprise SEQ. ID. NO.: 1, SEQ. ID. NO.: 3, SEQ. ID. NO.: 6, SEQ. ID. NO.: 7, SEQ. ID. NO.: 8, SEQ. ID. NO.: 9, SEQ. ID. NO.: 10 and/or SEQ. ID. NO.: 11.

20. The method of claim 16, wherein the individual is male.


SEQUENCE LISTING (11 sequences)

SEQ ID NO: 1 - DNA, 801 bp, Homo sapiens
cgccggatgtttgggtcttctggagctctctcctgctcttctggtatatccacaaacagtcttgtcaaaactggatcctgatacttcatacaattacttgcttcgggacctcttgagattcatttctcgtttcttcatttcctcaccaaatgctggagtccattgagtgagaaggggctagcctcagccatgtgtttactttggaggcctagtcaagagcttttctagaacatcctgtgggaatcagtggacttgccccaatattttctcattattttcacactattcactgttcccattcaatgcggctgctgagctattattttgctttctgtctaggcactggtaaccctggctggagcgccatcttggctgattctccatatcttattgtccttccrtgactcacatgtgctactgtccgttaaccatgctctttgtcacaaagcacaattctcagcttccccccagctcctactccttctccccaacacttgcacttacagaaatgtttccttcccatttcccttttctgtatctaccatcttttatctgccctaatatctacaaaaatatcctcagttctcttgaagtaaaaatggacacttttaaaattgttcatgtaagtatggatgtatatatctataaatttatgtatacatatttatgtattcatggatattggtgaataaatgtcagggtagccctactttttacacccgcttctctggtcacaatacagatgaactgtctgcatttgggtgggaggcagtgagagaagcatcgattgattggcatgat

SEQ ID NO: 2 - DNA, 601 bp, Homo sapiens
ggcaatctctatgttgggactaagaatttgatcctgttcatgctttgtctttctacttccaccttcagtgcctttttctgttcttttccggtgctctcttttctctcttttctcctctcccccatgcctcttgtggtatcatgctacctcatccttcctatcatgaccaaggcatttgatgaactcattcctgccagctcttctcagagtctgagtcagacaggaccacagggagaaaagactcttgaatcatgggacttacatttgggttaaagcacagaccaaatgctgtatgtaagarcctacctctgcaaggctgcccctgctcccactattgcacccatcctgccaggggccgactgggctgacaccatcattcccgtcactcccaccactcccctcccctcccagaattgcaatagtatgtcaaatctgggaccccgatcagggatctgggaagttgagaaagcatccagttggcaaactaagatatgggtttcaccctaagaaaagagttttaaatgctggctgtgcatgaaaattgcctgggaaattttagaagaatgtaagcgcctgtgccccatctgaggtcaatcaggtc

SEQ ID NO: 3 - DNA, 1057 bp, Homo sapiens
  misc_feature (383)..(383): n is a, c, g, or t
gaaaaaaaaataaggtcgagtaagcattaagaatcaagaattagtggatgatttctaaaatagtaaaagtagaatgtatgttattaccttctaaaaggaggcaaaagaaaattctgaaagcaggtaaaagaaaatttgaccaattccacgagtgaggaagagagataaaaaaagaagcaaagtttaaaaatgcagacaagaggcagaaaccaaactggttactctgacagagagaatgtaatgcatagaattgctaactacctggagaaatgaaaaggcaaaatggaaacagtgagggatcgtggatgtagcaaaggcgggaagcagccgtcgcacctcggctaggagaacaaagggaagaagttggaattcctaacagtttnggggaaagccttgcagagcaaaacccgtgagaatcaagtgatgctcagcccggttgggatcccagagatcagagcggggtccctgtgaggctgagtccctgacctctgaagaggggagggacgctgggcaacaggcactcacggccctaagagagtgtgatgaggccagttctgagaatcttggaaaaaactgaaaactgatctcaattcctgctactggatgggactgccactgccaacctgaagacacggacaggtgtctgacgggaaccgaatgaaagaaagcagaactaggacaaaccaggaaggagcaagtcccttccccctcctccagcctgcagcggcccaatcagcaggacctcacagggagccaggcagcaaggcaggcattgatttgcaacgtcccagcccagcatctcggggtggaggatggcaggtttgggactgagaaaccaaagctccatactcggcacagcctgccctgggctttgcttccacacactcccctctgtgagctttggaactcccaagacatccatgtttcaacttaacaagttgtggggcctctttcataagaaatggaggcattcccattctctcccttgaatgaggagatacaatgtcccagtcatcaatgctgccatctcctgaaaacaggccactgtccatctctg

SEQ ID NO: 4 - DNA, 601 bp, Homo sapiens
aactctctgatgctgagactaaaagtaattcatcttcctaaagactttctatctttgggagggtgacatttcctgtctggcttttctgatgattcttacaacttaattacttttggcatgtccagttgcttgctttatttactacattgtataggtcaaacacttgaacctcaggtttatgtttgttttatcaaacttagagaatataattattcagagagtaattttcagattagcacaaacatacattttctactagagagtatctattcttaaagctcacttgtgagacattatattyttcctgttgtgaggatgagatggcattctctttgggatctaaattgcttctgaggcaggcacttttgtgtattgttatctctataccccgaagaacaactggagtgcaggaaaagcagggttttagacaaatgaaagtgggatatgccgtctgggctgatggctacgcttggcatcaccaaatgggagacttaagagccttctgcagtcacggctgcagtggctgattattcttccagccatgtctcttccttccttcttctcttctcttctctcctcttctctcctcttctctcttctc

SEQ ID NO: 5 - DNA, 996 bp, Homo sapiens
acacttggacggcacgtagtcggcgtaggtctctgtgtgccccagctcctccgcctcctcctcctcagcctcgtcctcctcctctggctggctctgcacactctgctcctgcgtgcgtggcgggagctcagcggagactgcaggcctgagcggccgcgggcactgggctcccccaggggcaggggccaggctgaccatgggcagctgagtgacccaggggtgggagacggctctgtctgcccctgcagcaaggtgctcccccagggcctccatctcctcagctataaaatgggcactcccagcttctaggccagccaagcgctccccacaatggagatgcctgtgctgagccctctccttaggtcccatgagagggcaggggagggagctctcggctgggcccagctcacctggtagctgacgagaagcggggtgctgtgggagggcagaaagtcctcgaagcctgcaaacggcctgctgagctggaacagctggaagggragcaggatgtcagcccgggccagacgggacaggtcacagctggtgggccctcgcagcctggccaccagagggaggctccccactgccccgtcctcgcctccccatcctcgctccccagggccctgggtcttgctctcctgctgttctcagggtgaccctggtgcctcccaagcctccccagtgggcacctccctggggcctaaggctcgctcctcctggccagcacctggctgccttctcactgatggccagtgtgcctgcctggagtgcccgggaggaaggtgcccggctagttctgacagacaacacagcttggtcccgcgggagctgccaggaaggctgtggacaggggtctgacaggagagacccactgtgtgccagccccatgcagtgcaggaggccctcgggggcctgggagacaaggggttgggagctccctgtcgagggggaagaggagacagaggcagtgggggcgggcgggcctgggccttgtcc

SEQ ID NO: 6 - DNA, 601 bp, Homo sapiens
aaggactgcagtccaggaggagggaacacaccagtggatgtggcccaggggttgaaatcgggaagggtggttctccaagaagaatccaggatctgtcaccaaaagagcaactctagtgaggcaacagaaacaacatatgttggcataattcataatgatgatgtttccgttattgggaaccctggggagacaggattttgtgtttttggagtgattttcttgactctctttaatgcctgccccatagatagccagcttctcatgagactgtttcttgtttctctttcttctctgccacagrcagggccgaggctgagccccaacccaagatgagcctttggtttagtagcctgggacctggacatttggaaagagacagatcagactggaagcctctccatccggcttccacagattacctttgtgttacttccttttcccctagtcaagacttgctttaagttttaaggtggtaggaaggagatattttagtaaagtatccgatgtatctttttggtgctatgctaattattaccaagagcctcagtggttttgactgcagcctccacattttgcaccaccatgatggggtttagcaata

SEQ ID NO: 7 - DNA, 951 bp, Homo sapiens
ggaaaactgtaatctcaaactacttccagagaagtacaactatcattcctgtctactctccactactttgtcagagcttttaaacattttactagcttgcaattttactgcgtggaagacttgcaagtcatctgcaagggtgaggtttccctgtgtgctcactcattttaggtcatggagaagtattcatttgtcctgatggttggttttggtctccgggtttcctgtctagacccgctaaatgcctactggtctcatggctgcattttgaagtcatcaggttagacttggggcatgtttgaagtaggatctgaatccaggggaggcaatggcttcctgacaagggtagtttctcaaaacctgggattttcactcctgcaagttaggagtgggcatgatgagaatataaaatcatgaagcaactcaaatcagacttacttagtatactagaacaaggagggtatcccttgatgttggcagggacaaagaaagtgaatttkctgtttaaatatcattaaggaagtgtgtggaagtcagtcacgaaggggtggtgcagactaaaacaccaaaaggttcaaatgagctctgagtcaatcaatgcgtgattaataagtaaggaatcacttcagggaattaggggtgtttcttttattaaaaaaaattaaaaatagaactatcatacaatccagcaatcttactactgggtatatatccaaaggaaataaaatgagtatgcggaagagatatctgcacccccccaccccgtttattgccgcactattcataatagccaagatgtagaacaaacctaagtgtccaacagtgaatgaatgggtgaagaagatgtggtgcatatacacaatggaatactattcagctatataaaagaaggaaatgttgttctttgtgacaacatgaatgaatctggaggacgctatgctaaatgaaata

SEQ ID NO: 8 - DNA, 601 bp, Homo sapiens
  misc_feature (301)..(301): n is a, c, g, or t
tgaataggcaactatatgtctaacagttttaagttaaattttttaaaagtagcacatagttataatggtcattttacatattttgccaaataattatcctcacttgaaagaggttattaattcttggcttacaaagggaaaactgttttggttttcaagcaccaaagtttaatacatccagttatctcaagggaatgagggatctgttcactggcaatatcttttttgttttccttttctttttgtgattgaaagtaaggataagggaactgatgtagagacaaggacatgctgaagtacngaggaggtattggaaaatttctttgccatagccaaccaaattttggaataaatggtactaaaatatctggatgatttgtagtaaaaaaaacattgcttgcttgtctgtctacttgattaataccccttgcctttgtcttttggcttataatcctacatttggaataaataagcctctttccatattttattcctttctgccttcaggaaagtacatcttccctgttttggaaagaaacaaactaactcctggacttctccccagcccttcgtttgagcctgtttccctttccaaggactt

SEQ ID NO: 9 - DNA, 746 bp, Homo sapiens
tgagtgtccccatctgggtccttgctcagtgagagccagactttctgctataaccaggccctgaaygagagagaaccagagtcatgtgctgtgcaccaaatgcagtgggaagacacgagggatgcctcggtgaccttcttttcaaacaggagtctttcgctggtaacctgcctctgaccttccatcagccagatgttatcttcccaaggcaacagtgttccagaattctttctgaggtggccctggcagcttctgggtccttctattctgatttccagttttctttttccatttatttatttatttatttatttatttatttatttatttattaataagggtcttgctttgttgcccaggctggagtgcagtggcacgaacacagctcactgcagcctcaaactcctgggctccagtgatcctcccgccccagcctcctgtgtagctgggactacgggcatgcaccaccatgcctggctaactttttatagagatggggtctcactatgttgcccaagctggtctcaaactagcagactcagtgattctcctgcctcagctttctgagtagctgggaccacaagtgagcaccattatgcctggctaatttttatagagatagggtctcactatgttgcccaagctggcctagactagtgggacagtgactcactcactcggactgatcgctggcatgtgtgattagagtgtgcatgtatgcaaccctgcaggaaggccgccgtggt

SEQ ID NO: 10 - DNA, 805 bp, Homo sapiens
ctgctgtgctggtgggagggccactggccaacctgctctcacccagcttgggctggccatagtgccaggcagttgccctcagctcctctgcccattccagccatggaggtggctaaccttgcaggctccagggcgtgatgggagtgtccaggaggcagaggccagagggggtcatgctgtagggggagttcctctctcagaccccatctgtgcctcccccgctgcgtatctgggtgacatggatgccaacgggcaggagcaagggctgggaaaggctgcggaggggccctcggctggtctggtggggagcggggtcactgtgtctctgctgcctccctgggggatccagcgtggggatgggacatggagctgggcacctctcctggatgcccctgggctgatccccctcgatgccctgacgagagccctggcccagcgtctgagaaggagtgggagctgggcccgggtgggggcaggagccggcatcgctctcagcactgccccacactccccagcctgcctgctgaggccatgtcggactacgagaacgatgacgagtgctggarcgtcctggaaggcttccgggtgacgctcacctcggtcatcgacccctcacgcatcacaccttacctgcggcagtgcaaggtcctgaaccccgatgatgaggagcaggtgctcagcgaccccaacctggtcatccgcaaacggaaagtgggtcagtgttgccccgcggggccccagccccaaacccaccacagaatcagctcggaagtggctgcacgctttgccaccctgacccttaacc

SEQ ID NO: 11 - DNA, 801 bp, Homo sapiens
ttatatgcctcggtttcctcatctaataaaaaagcaaaaagtaattaggctgagttacctatgcttggtctgttgaagtagtttgaatttttctgaatcaggatgaatgcaaattgcctcattatttcactaattaataatgtctgcttggaaatgcaactatcagtgcacaataaatgataaagcttataattatagatgagagcaattgcttctttttttgttcttaccttcatttcagtttatcttcctgagctatagctacctctgtaagggttaggaaaacatttaaaatatctaaacagtgattcctttctcaagcgttcggaaattgatgaatcatcttactctgcagcattcaaagtaactgtgtgcttgacacataggaggaaaagtgggcyctttgaaatgtgagcagagcaccgatcgtgtcacctgcccggcgcgtgacctgttcactttccacctgaccctctgtgcccaatgtcacctggagcctgaaacctggaagggaaaagtccttgactggagtcagtggagagaaaatcatcacctgcttgggcacaaacatgcctggtgactgcgtcactgtctgttaagtgggatcagctaatagttgcacagcgggccttcaaagcatcacggcagaactgatgaagttcctgtaatgcattccctgtgtgaaggccttggactttagagctcaagtgcgccaggagcattttgagcttgccgacaggctctcccatggattctatttggttatacggcagtggaaaagaattgactttcattttcgtc